SEQUENCE LISTING (3 sequences)

SEQ ID NO: 1 - protein, 20 aa, Hepatitis C virus
EKALDCQIYGACYSIEFLDL

SEQ ID NO: 2 - DNA, 60 bp, Hepatitis C virus
gaaaaagctcttgattgtcaaatttatggtgcttgttattctattgaatttcttgatctt

SEQ ID NO: 3 - DNA, 60 bp, Hepatitis C virus
gagaaggcvttvgactgccagatmtacggvgcvtgctacagvatmgagttcttvgacttv

(19) United States
(12) Patent Application Publication
(10) Pub. No.: US 2007/0128625 A1
(43) Pub. Date: Jun. 7, 2007

(54) Drug-resistant mutants of hepatitis C virus

(75) Inventors: Nina Boddeker, Berkeley, CA (US); Johan Neyts, Kessel-Lo (BE); I-Hung Shih, San Francisco, CA (US); Inge Vliegen, Tongeren (BE); Weidong Zhong, San Ramon, CA (US)

Correspondence Address: Intellectual Property Department;Gilead Sciences, Inc., 333 Lakeside Drive, Foster City, CA, 94404, US

(73) Assignee: GILEAD SCIENCES, LLC

(21) Appl. No.: 11/491,756

(22) Filed: Jul. 24, 2006

Related U.S. Application Data
(60) Provisional application No. 60/702,534, filed on Jul. 25, 2005.

(51) Int. Cl. C12Q 1/70; C12Q 1/68

(52) U.S. Cl. 435/6; 435/5

(57) ABSTRACT

The present invention provides nucleotides, peptides, HCV mutants, and cell lines containing mutations associated with drug resistance. In addition, the present invention provides methods for screening for therapeutic compounds capable of inhibiting HCV as well as methods for inhibiting HCV, e.g., by targeting specific binding sites associated with HCV drug resistance.

### FIELD OF THE INVENTION

This invention relates in general to drug resistant mutants of hepatitis C virus, specifically mutations implicating interaction sites between anti-HCV therapeutic compounds and hepatitis C virus.

### BACKGROUND OF THE INVENTION

In 2004, it was estimated that there were over 170 million individuals, worldwide, infected with the Hepatitis C virus (HCV, Semin. Liver Dis. 2000, 20, 1). In the late 1990s, HCV infection was shown to be responsible for more than half of chronic hepatitis cases, and for 30% of end-stage liver disease (Hepatology, 1997, 26 (3 suppl. 1), 15S-20S). Of patients acutely infected with HCV, approximately 85% go on to develop slow, progressive liver disease and between 20 to 30% eventually develop cirrhosis. HCV infection is also the most common risk factor associated with the development of hepatocellular carcinoma and is a main causal agent necessitating liver transplants (Transplant Mt. 2002, 15, 61).

The current standard-of-care treatment for HCV infection is interferon-α (or its PEG-derivatized equivalent) in combination with ribavirin, a regimen that produces sustained virologic response in only 40% of people infected with the HCV genotype 1. This regimen has significant side effects leading an unacceptable number of patients to discontinue treatment (Hepatology, 2002, 2, 205). There is a clear need for novel therapies that are both more effective, and more tolerable to treat patients in an epidemic which is represented by approximately 5-fold more infected individuals than the HIV epidemic.

Drug discovery research to find novel HCV therapeutics has been hampered by a lack of direct viral infection techniques or a simple small animal model of infection. The development of replicons, which are autonomously replicating RNA molecules derived partially or fully from wildtype HCV, have been a valuable boost to HCV research (Science 1999, 285, 110). Such replicons can be transfected into permissive cell lines, such as Huh-7 and others. The resulting replicon-carrying cell lines can be used for testing potential HCV inhibitors and for identifying resistant mutants to known HCV inhibitors.

There is a need in the art to identify mutations associated with drug resistant, which could be useful for genotyping of HCV infection and for understanding interactions between therapeutic compounds and HCV, thus developing therapeutic compounds with better targeting and inhibition capability to HCV infection.

### SUMMARY OF THE INVENTION

It is the discovery of the present invention that certain mutations within HCV are associated with drug resistance, thus implicating specific active sites involved in interactions between HCV and therapeutic compounds. Accordingly the present invention provides nucleotides, peptides, HCV mutants, and cell lines containing mutations associated with drug resistance. In addition, the present invention provides methods for screening for therapeutic compounds capable of inhibiting HCV as well as methods for inhibiting HCV, e.g., by targeting specific binding sites associated with HCV drug resistance.

In one embodiment, the present invention provides a method for inhibiting HCV activity. The method comprises contacting HCV with a therapeutic entity capable of interacting specifically with a HCV binding site containing one or more amino acids selected from the group consisting of 1) Q581, A391, M582, and C432 within NS3 region, 2) V24 within NS4A region, 3) L4, Q93, and L78 within NS4B region, 4) M416, E441, and V362 within NS5A-region, and 5) C316, C445, Y448, and Y452 within NS5B region.

In another embodiment, the present invention provides a nucleotide probe comprising a sequence capable of detecting a mutation within HCV, wherein the mutation corresponds to 1) Q581E, A39 IV, M582L, or C432S within NS3 region, 2) V24A within NS4A region, 3) L4P, Q93, or L78T within NS4B region, 4) M416T, E441G, or V362A within NS5A region, or 5) C316Y, C445F, Y448H, or Y452H within NS5B region.

In yet another embodiment, the present invention provides a cell line containing HCV replicon comprising at least one mutation selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, or 5) C316Y, C445F, Y448H, and Y452H within NS5B region.

In yet another embodiment, the present invention provides an HCV replicon containing at least one mutation selected from the group consisting of 1) Q581E, A39 1V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region.

In yet another embodiment, the present invention provides a method for determining treatment for an HCV infection comprising detecting the presence or absence of a HCV mutation selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region.

In yet another embodiment, the present invention provides an antibody capable of specifically binding to a HCV binding site containing at least one amino acid selected from the group consisting of 1) Q581, A391, M582, and C432 within NS3 region, 2) V24 within NS4A region, 3) L4, Q93, and L78 within NS4B region, 4) M416, E441, and V362 within NS5A region, and 5) C316, C445, Y448, and Y452 within NS5B region.

In yet another embodiment, the present invention provides an antibody capable of specifically binding to an HCV epitope containing at least one mutation selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region.

In still another embodiment, the present invention provides a method for screening potential therapeutic entities comprising contacting a testing entity with an HCV replicon comprising at least one mutation selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region, and determining anti-HCV activity of the testing entity.

### BRIEF DESCRIPTION OF THE DRAWINGS

FIG. 1. Mutations present in drug resistant HCV replicons.

FIG. 2. Mutations conferring resistance to imidazopyridine compounds

FIG. 3. Sequences of NS5B region carrying resistance mutations. Part A) shows the amino acid sequence of the relevant region of NS5B with mutations at positions 445 and 448 underlined. Part B) shows nucleic acid sequences that could encode the amino acid sequence of A). Each position may have redundancies, as indicated in the second line of part B). The second line shows alternative nucleic acid bases at each appropriate position; X and Y represent more than one alternative base as indicated in Part B).

### DETAILED DESCRIPTION

It is the discovery of the present invention that certain mutations within HCV are associated with drug resistance, thus implicating specific sites involved in interactions between HCV and therapeutic compounds. Accordingly the present invention provides nucleotides, peptides, HCV mutants, and cell lines containing mutations associated with drug resistance. In addition, the present invention provides methods for screening for therapeutic compounds capable of inhibiting HCV as well as methods for inhibiting HCV, e.g., by targeting specific binding sites associated with HCV drug resistance.

According to one aspect of the present invention, it provides a method for inhibiting HCV activity by contacting HCV with a therapeutic entity, e.g., chemical or biological molecules capable of interacting specifically with a HCV binding site or active site. In general, the HCV binding site or active site of the present invention contains one or more amino acids involved in HCV drug resistance, especially amino acids mutated in response to anti-HCV drug treatment. In one embodiment, the HCV binding site or active site of the present invention contains one or more amino acids within NS3, NS4A, NS4B, NS5A, and/or NS5B and are likely to mutate in response to anti-HCV drug treatment. In another embodiment, the HCV binding site or active site of the present invention contains one or more amino acids within NS5B and are likely to mutate in response to anti-HCV drug treatment. In yet another embodiment, the HCV binding site or active site of the present invention contains one or more amino acids within NS5A and NS5B regions and are likely to mutate in response to anti-HCV drug treatment.

In yet another embodiment, the HCV binding site or active site of the present invention contains one or more amino acids selected from the group consisting of 1) Q581, A391, M582, and C432 within NS3 region, 2) V24 within NS4A region, 3) L4, Q93, and L78 within NS4B region, 4) M416, E441, and V362 within NS5A region, and 5) C316, C445, Y448, and Y452 within NS5B region. In yet another embodiment, the HCV binding site or active site of the present invention contains one or more amino acids selected from the group consisting of 1) V24 within NS4A region, 2) M416 and E441 within NS5A region, and 3) C316, C445, Y448, and Y452 within NS5B region. In yet another embodiment, the HCV binding site or active site of the present invention contains at least C316 within NS5B region. In yet another embodiment, the HCV binding site or active site of the present invention contains one or more amino acids selected from the group consisting of C316, C445, Y448, and Y452 within NS5B region.

According to the present invention, any suitable therapeutic entity capable of interacting specifically with the HCV binding site or active site of the present invention can be used to inhibit HCV activity. For example, certain imidazopyridine compounds described in WO 2003/005286 and WO 2005/063744 can be used according to the present invention to inhibit HCV activity. A representative imidazopyridine structure is shown as Formula A

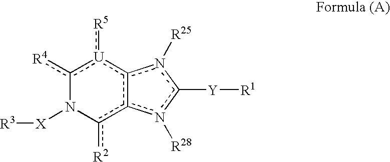

wherein:
the dotted lines represent an optional double bond, provided that no two double bonds are adjacent to one another, and that the dotted lines represent at least 3, optionally 4 double bonds;
U is N or C;
R¹ is selected from hydrogen; aryl unsubstituted or substituted with one or more R⁶, heterocyclic ring unsubstituted or substituted with one or more R⁶, C₃₋₁₀ cycloalkyl unsubstituted or substituted with one or more R⁶ and C₄₋₁₀ cycloalkenyl unsubstituted or substituted with one or more R⁶;
Y is selected from the group consisting of a single bond, O; S(O)ₘ; NR¹¹; and a divalent, saturated or unsaturated, substituted or unsubstituted C₁-C₁₀ hydrocarbon group optionally including one or more heteroatoms in the main chain, said heteroatoms being selected from the groups consisting of O, S, and N; such as C₁₋₆ alkylene, C₂₋₆ alkenylene, C₂₋₆ alkynylene, —O(CH₂)₁₋₅−, —(CH₂)₁₋₄—O—(CH₂)₁₋₄—, —S—(CH₂)₁₋₅—, —(CH₂)₁₋₄—S—(CH₂)₁₋₄—, —NR¹¹—(CH₂)₁₋₅—, —(CH₂)₁₋₄—NR¹¹—(CH₂)₁₋₄— and C₃₋₁₀ cycloalkylidene;
each R² and R⁴ is independently selected from the group consisting of hydrogen C₁₋₁₈ alkyl; C₂₋₁₈ alkenyl; C₂₋₁₈ alkynyl; C₁₋₁₈ alkoxy; C₁₋₁₈ alkylthio; halogen; OH; CN; NO₂; NR⁷R⁸; OCF₃; haloalkyl; C(═O)R⁹; C(═S)R⁹; SH; aryl; aryloxy; arylthio; arylalkyl ; C₁₋₁₈ hydroxyalkyl; C₃₋₁₀ cycloalkyl; C₃₋₁₀ cycloalkyloxy; C₃₋₁₀ cycloalkylthio; C₃₋₁₀ cycloalkenyl; C₃₋₁₀ cycloalkynyl; 5 or 6 membered heterocyclic, oxyheterocyclic or thioheterocyclic ring; or, when one of R²⁵ or R²⁶ is different from hydrogen, either R² or R⁴ is selected from (═O), (═S), and (═NR²⁷);
X is selected from the group consisting of a divalent, saturated or unsaturated, substituted or unsubstituted C₁-C₁₀ hydrocarbon group optionally including one or more heteroatoms in the main chain (provided that the heteroatom is not linked to N of the nucleus), said heteroatoms being selected from the group consisting of O, S, and N; such as C₁₋₆ alkylene, (for example —CH₂—, —CH(CH₃)—, —CH₂—CH₂—, —CH₂—CH₂—CH₂—, —CH₂—CH₂—CH₂—CH₂), —(CH₂)₂₋₄—O—(CH₂)₂₋₄—, —(CH₂)₂₋₄—S—(CH₂)₂₋₄—, —(CH₂)₂₋₄—NR¹⁰—(CH₂)₂₋₄—, C₃₋₁₀ cycloalkylidene, C₂₋₆ alkenylene (such as —CH═CH—CH₂—), C₂₋₆ alkynylene;
m is any integer from 0 to 2;
R³ is selected from the group consisting of aryl; aryloxy; arylthio; aryl-NR¹⁰—; 5 or 6 membered heterocyclic, oxyheterocyclic or thioheterocyclic ring; and each of said aryl, aryloxy, arylthio, aryl-NR¹⁰—, 5 or 6 membered heterocyclic, oxyheterocyclic or thioheterocyclic ring is optionally substituted with one or more R¹⁷; C₃₋₁₀ cycloalkyl, oxycycloalkyl or thiocycloalkyl; C₄₋₁₀ cycloalkenyl with the proviso that the double bond cannot be adjacent to a nitrogen; H with the proviso that if X is an alkylene, an alkenylene or an alkynylene, then X comprises at least 5 carbon atoms;
R⁵ is independently selected from the group consisting of hydrogen; C₁₋₁₈ alkyl; C₂₋₁₈ alkenyl; C₂₋₁₈ alkynyl; C₁₋₁₈ alkoxy; C₁₋₁₈ alkylthio; halogen; OH; CN; NO₂; NR⁷R⁸; OCF₃; haloalkyl; C(═O)R⁹; C(═S)R⁹; SH; aryl; aryloxy; arylthio; arylalkyl; C₁₋₁₈ hydroxyalkyl; C₃₋₁₀ cycloalkyl; C₃₋₁₀ cycloalkyloxy; C₃₋₁₀ cycloalkylthio; C₃₋₁₀ cycloalkenyl; C₃₋₁₀ cycloalkynyl; 5 or 6 membered heterocyclic, oxyheterocyclic or thioheterocyclic ring;
each R⁶ and R¹⁷ is independently selected from the group consisting of hydrogen; C₁₋₁₈ alkyl; C₂₋₁₈ alkenyl; C₂₋₁₈ alkynyl; C₁₋₁₈ alkoxy; C₁₋₁₈ alkylthio; cycloalkyl, C₃₋₁₀ cycloalkenyl or C₃₋₁₀ cycloalkynyl; halogen; OH; CN; NO₂; NR⁷R⁸; OCF₃; haloalkyl; C(═O)R¹⁸; C(═S)R¹⁸; SH; aryl; aryloxy; arylthio; arylalkyl; arylalkyloxy (optionally a oxybenzyl); arylalkylthio (optionally a benzylthio); 5 or 6 membered heterocyclic, oxyheterocyclic or thioheterocyclic ring; C₁₋₁₈ hydroxyalkyl; and each of said aryl, aryloxy, arylthio, arylalkyl, arylalkyloxy (optionally a oxybenzyl), arylalkylthio (optionally a benzylthio), 5 or 6 membered heterocyclic, oxyheterocyclic or thioheterocyclic ring, C₁₋₁₈ hydroxyalkyl is optionally substituted with 1 or more R¹⁹;
each R⁷ and R⁸ is independently selected from the group consisting of H; C₁₋₁₈ alkyl; C₁₋₁₈ alkenyl; aryl; C₃₋₁₀ cycloalkyl; C₄₋₁₀ cycloalkenyl; 5-6 membered heterocyclic ring; C(═O)R₁₂; C(═S)R¹²; an amino acid residue linked through a carboxyl group thereof; alternatively, R⁷ and R⁸, together with the nitrogen to which they are attached, combine to form a 5-6 membered heterocyclic ring;
each R⁹ and R¹⁸ is independently selected from the group consisting of H; OH; C₁₋₁₈ alkyl; C₂₋₁₈ alkenyl; C₃₋₁₀ cycloalkyl; C₄₋₁₀ cycloalkenyl; C₁₋₁₈ alkoxy; NR¹⁵R¹⁶; aryl an amino acid residue linked through an amino group thereof;
each R¹⁰ and R¹¹ is independently selected from the group the group consisting of H; C₁₋₁₈ alkyl; C₁₋₁₈ alkenyl; C₃₋₁₀ cycloalkyl; C₄₋₁₀ cycloalkenyl; aryl; C(═O)R₁₂; 5-6 membered heterocyclin ring; an amino acid residue linked through a carboxyl group thereof;
R¹² is independently selected from the group consisting of H; C₁₋₁₈ alkyl; C₂₋₁₈ alkenyl; aryl; C₃₋₁₀ cycloalkyl; C₄₋₁₀ cycloalkenyl; an amino acid residue linked through an amino group thereof;
each R¹³ and R¹⁴ is independently selected from the group consisting of H; C₁₋₁₈ alkyl; C₂₋₁₈ alkenyl; aryl; C₃₋₁₀ cycloalkyl; C₄₋₁₀ cycloalkenyl; C(═O) C(═S)R¹²; an amino acid residue linked through a carboxyl group thereof;
each R¹⁵ and R¹⁶ is independently selected from the group consisting of H; C₁₋₁₈ alkyl; C₂₋₁₈ alkenyl; C₂₋₁₈ alkynyl; aryl; C₃₋₁₀ cycloalkyl; C₄₋₁₀ cycloalkenyl; an amino acid residue linked through a carboxyl group thereof;
R¹⁹ is independently selected from the group consisting of H; C₁₋₁₈ alkyl, preferably C₁₋₆ alkyl; C₂₋₁₈ alkenyl; C₂₋₁₈ alkynyl; C₁₋₁₈ alkoxy, preferably C₁₋₆ alkoxy; C1-18 alkylthio; C₃₋₁₀ cycloalkyl; C₄₋₁₀ cycloalkenyl; C₄₋₁₀ cycloalkynyl; halogen; OH; CN; NO₂; NR²⁰R²¹; OCF₃; haloalkyl; C(═O)R²²; C(═S)R²²; SH; C(═O)N(C₁₋₆ alkyl), N(H)S(O)(O)(C₁₋₆ alkyl); aryl; aryloxy; arylthio; arylalkyl; and each of said aryl, aryloxy, arylthio, arylalkyl substituted with 1 or more halogens, particularly a phenyl substituted with 1-2 halogens; hydroxyalkyl; 5 or 6 membered heterocyclic, oxyheterocyclic or thioheterocyclic ring each unsubstituted or substituted with 1 or more halogens;
each R²⁰ and R²¹ is independently selected from the group consisting of H; C₁₋₁₈ alkyl, preferably C₁₋₆ alkyl; C₂₋₁₈ alkenyl; C₂₋₁₈ alkynyl; aryl; C₃₋₁₀ cycloalkyl; C₄₋₁₀ cycloalkenyl; C(═O)R¹², C(═S)R¹²;
R²² is independently selected from H; OH; C₁₋₁₈ alkyl; C₂₋₁₈ alkenyl; C₁₋₁₈ alkoxy; NR²³R²⁴; aryl; C₃₋₁₀ cycloalkyl, ; C₄₋₁₀ cycloalkenyl;
each R²³ and R²⁴ is independently selected from the group the group consisting of H; C₁₋₁₈ alkyl, preferably C₂₋₃ alkyl, wherein C₂₋₃ alkyl taken together with N of R²² can form a saturated heterocycle, which heterocycle is optionally substituted with OH or aryl or an amino acid residue;
each R²⁵ or R²⁶, selected from the group consisting of H, C₁₋₁₈ alkyl, preferably C₁₋₄ alkyl; C₃₋₁₀ cycloalkyl, such as C₅₋₁₀ bicycloalkyl; C₃₋₁₀ cycloalkenyl; (C₃₋₈ cycloalkyl)-C₁₋₃ alkyl; aryl, such as phenyl; 5 or 6 membered heterocyclic ring, such as pyridyl; alkylaryl, such as benzyl; and each of said C₁₋₁₈ alkyl, preferably C₁₋₄ alkyl, C₃₋₁₀ cycloalkyl, C₃₋₁₀ cycloalkenyl, (C₃₋₈ cycloalkyl)-C₁₋₃ alkyl, C₅₋₁₀ bicycloalkyl, adamantyl, phenyl, pyridyl and benzyl is optionally substituted with 1-4 of each of C₁₋₆ alkyl, C₁₋₆ alkoxy, halo, CH₂OH, oxybenzyl, and OH; and heterocyclic ring having 3 to 7 carbon atoms, preferably a saturated heterocyclic ring wherein the heteroatoms are S, S(O), or S(O)₂ separated from the imidazopyridyl ring nitrogen atom by at least 2 heterocyclic ring carbon atoms. Provided that either R²⁵ or R²⁶ is hydrogen. Typically R²⁵ or R²⁶ is cyclopentyl or cyclohexyl; provided that if the compound is substituted at R²⁵ or R²⁶, either R² or R⁴ is selected from (═O), (═S), and (═NR²⁷); and
R²⁷ is selected from the group consisting of H, C₁₋₁₈ alkyl, C₃₋₁₀ cycloalkyl, (C₃₋₁₀ cycloalkyl)-C₁₋₆ alkyl; aryl; arylalkyl, such as benzyl.

A representative subset of highly active imidazopyridine compounds of Formula (A) is shown below. The compounds are effective inhibitors of HCV replicons and are currently under development as human HCV therapeutics.

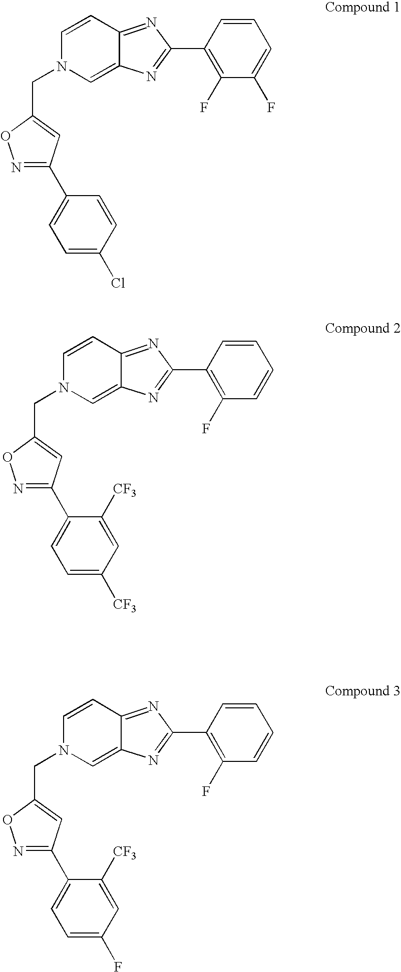

According to another aspect of the present invention, it provides antibodies, e.g., monoclonal or humanized monoclonal antibodies capable of specifically binding to the HCV binding site or active site of the present invention. In one embodiment, the antibodies of the present invention are capable of specifically binding to one or more epitopes within the HCV binding site or active site of the present invention. In another embodiment, the antibodies of the present invention are capable of specifically binding to at least one epitope within the HCV binding site or active site of the NS5B region of the present invention. In yet another embodiment, the antibodies of the present invention are capable of specifically binding to at least one epitope containing one or more amino acids selected from the group consisting of 1) Q581, A391, M582, and C432 within NS3 region, 2) V24 within NS4A region, 3) L4, Q93, and L78 within NS4B region, 4) M416, E441, and V362 within NS5A region, and 5) C316, C445, Y448, and Y452 within NS5B region. In yet another embodiment, the antibodies of the present invention are capable of specifically binding to at least one epitope containing one or more amino acids selected from the group consisting of 1) V24 within NS4A region, 2) M416 and E441 within NS5A region, and 3) C316, C445, Y448, and Y452 within NS5B region. In yet another embodiment, the antibodies of the present invention are capable of specifically binding to at least one epitope containing at least C316 within NS5B region. In yet another embodiment, the antibodies of the present invention are capable of specifically binding to at least one epitope containing one or more amino acids selected from the group consisting of C316, C445, Y448, and Y452 within NS5B region.

According to another embodiment of the present invention, it provides antibodies, e.g., monoclonal or humanized monoclonal antibodies capable of specifically binding to one or more epitopes containing one or more mutations associated with HCV drug resistance. In one embodiment, the antibodies of the present invention are capable of specifically binding to an epitope containing one or more mutations associated with HCV drug resistance and located within NS5B region. In another embodiment, the antibodies of the present invention are capable of specifically binding to an epitope containing one or more mutations selected from the group consisting of 1) Q581E, A391V, M582L, and C432S within NS3 region, 2) V24A within NS4A region, 3) L4P, Q93, and L78T within NS4B region, 4) M416T, E441 G, and V362A within NS5A region, and 5) C316Y, C445F, Y448H, and Y452H within NS5B region.

According to the present invention, the antibodies of the present invention can be used to inhibit HCV activity, thus treat HCV infection, e.g., by administering to a subject in need of such treatment an effective amount of the antibodies of the present invention. In one embodiment, antibodies capable of specifically binding to an epitope containing one or more mutations associated with HCV drug resistance are used to treat subjects infected with drug resistant HCV.

According to another aspect of the present invention, it provides nucleotide probes capable of detecting one or more mutations associated with HCV drug resistance. In general, the nucleotide probes of the present invention can be oligonucleotide probes or polynucleotide probes. They can be any size that is compatible for a particular detection method, e.g., PCR, etc and capable of detecting one or more mutations under a particular detecting condition, e.g., hybridization stringency conditions. For example, the nucleotide probes of the present invention can be from about 10 nucleotides to about 15, 20, 25, 30, 35, 40, 45, 50, 55, or 60 nucleotides or from about 100 nucleotides to about 200, 300, 400, 500, 600, 1000, or 2000 nucleotides. In one embodiment, the nucleotide probes of the present invention is labeled with one or more detectable groups or entities, e.g., groups either directly detectable via any suitable means or detectable via any suitable chemical or enzymatic reactions. In another embodiment, the nucleotide probes of the present invention are capable of detecting one or more mutations corresponding to 1) Q581 E, A391 V, M582L, or C432S within NS3 region, 2) V24A within NS4A region, 3) L4P, Q93, or L78T within NS4B region, 4) M416T, E441G, or V362A within NS5A region, or 5) C316Y, C445F, Y448H, or Y452H within NS5B region.

According to the present invention, the nucleotide probes of the present invention can be used for genotyping of HCV infection, thus determining treatment for an HCV infection. For example, a subject, e.g., human under anti-HCV treatment may develop drug resistance. Detection of such resistance can be useful for selection of particular anti-HCV therapeutic compounds or combinations thereof and for determining the duration and dosage of using these compounds with respect to the subject under treatment. Alternatively, a subject, e.g., human may be infected with drug resistant HCV and can be tested for HCV drug resistance prior to any anti-HCV treatment so that proper anti-HCV compounds can be selected to treat the infected subject.

In one embodiment, one or more nucleotide probes of the present invention are used to detect one or more mutations corresponding to 1) Q581E, A391V, M582L, or C432S within NS3 region, 2) V24A within NS4A region, 3) L4P, Q93, or L78T within NS4B region, 4) M416T, E441G, or V362A within NS5A region, or 5) C316Y, C445F, Y448H, or Y452H within NS5B region. In another embodiment, one or more nucleotide probes of the present invention are used to detect at least two or three mutations selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region.

According to another aspect of the present invention, it provides cell lines containing HCV replicons with one or more mutations associated with HCV drug resistance, e.g., mutations selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region. In addition, the present invention provides HCV replicons containing one or more mutations associated with HCV drug resistance, e.g., mutations selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region.

According to another aspect of the present invention, it provides methods for screening potential therapeutic entities using cell lines or HCV replicons containing one or more mutations associated with HCV drug resistance. In one embodiment, the method of the present invention comprises contacting testing compounds or entities with cell lines or HCV replicons containing one or more mutations selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region and determining anti-HCV activity of the testing compounds or entities. In another embodiment, the method of the present invention comprises contacting testing compounds or entities with cell lines or HCV replicons containing one or more mutations selected from the group consisting of C316Y, C445F, Y448H, and Y452H within NS5B region and determining anti-HCV activity of the testing compounds or entities.

### EXAMPLES

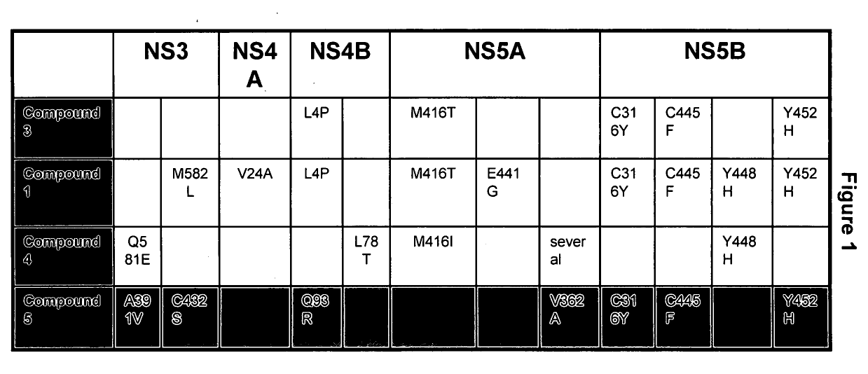

Mutant replicons resistant to the effects of Compounds 1, 2 or 3 were selected by growing Huh-luc7neo cells containing an HCV replicon in the presence of one of the compounds and in the simultaneous presence of G418, a drug that selects for cells carrying an HCV replicon. RNA from resistant cell clones was isolated and sequenced. Mutations were found at several sites within the HCV replicon, as summarized in FIG. 1. Mutations in the NS5B gene, encoding the viral RNA-dependent RNA polymerase (RdRp) were further studied.

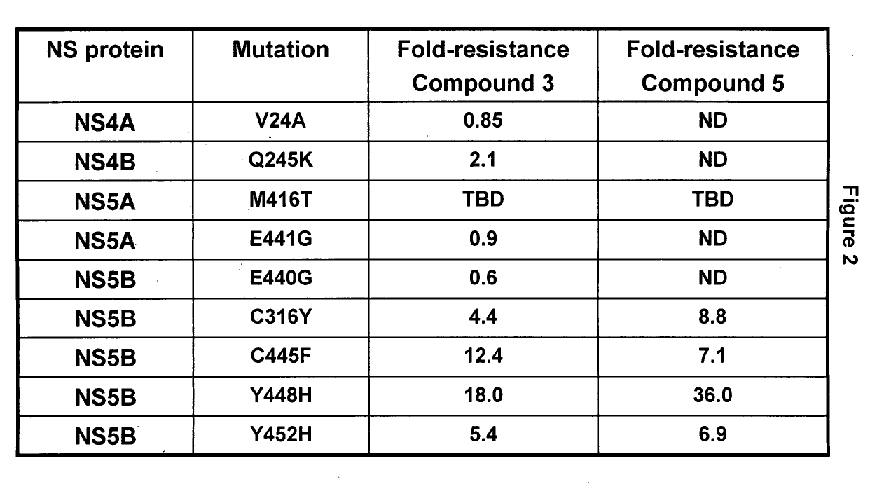

The mutations V24A, E441G, C316Y, C445F, Y448H, and Y452H were found to be sufficient for resistance when introduced into wildtype replicons. The combination of two mutations was found to be resistant at a higher drug level than either single mutation, as shown in FIG. 2.

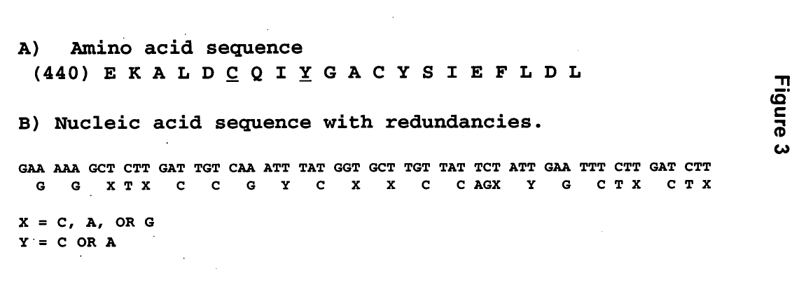

The sites of mutations C445F and Y448H can be visualized by using a model of the HCV RdRp protein. It is expected that other compounds that bind the same region of NS5B as the imidazopyridine compounds of WO 2004/005286 and WO 2005/063744 can also be affected by the same mutations. Thus, the resistance mutations shown in FIGS. 1-3 could also confer resistance to compounds that are outside the scope of WO 2004/005286 and WO 2005/063744 if such compounds bind the same region. Such binding would be evidenced by competing with imidazopyridine compounds for binding in assays such as are known to those of average skill in the art.

The sequence surrounding the NS5B mutations conferring resistance to imiazopyridine drugs may be useful as probes for detecting genotype in viral nucleic acid from patients. Such genotype information would be valuable in predicting clinical utility of any imidazopyridine drug or other drug known to bind to the same region in HCV NS5B. Thus the sequences shown in FIG. 4 would be useful as specific probes for tests or assay kits for determining the presence or absence of resistance mutations. Such sequences are also useful as reference sequences to be used in comparing the sequence of a test sample for purposes of predicting clinical efficacy of antiviral drugs. Kits and assay techniques to determine the genotype of a patient's virus are known to those skilled in the art and may be available commercially.

What is claimed:

1. A method for inhibiting HCV activity comprising contacting HCV with a therapeutic entity capable of interacting specifically with a HCV binding site containing one or more amino acids selected from the group consisting of 1) Q581, A391, M582, and C432 within NS3 region, 2) V24 within NS4A region, 3) L4, Q93, and L78 within NS4B region, 4) M416, E441, and V362 within NS5A region, and 5) C316, C445, Y448, and Y452 within NS5B region.

2. The method of claim 1, wherein the HCV binding site contains one or more amino acids selected from the group consisting of 1) V24 within NS4A region, 2) M416 and E441 within NS5A region, and 3) C316, C445, Y448, and Y452 within NS5B region.

3. The method of claim 1, wherein the HCV binding site contains at least C316 within NS5B region.

4. The method of claim 1, wherein the HCV binding site contains one or more amino acids selected from the group consisting of C316, C445, Y448, and Y452 within NS5B region.

5. The method of claim 1, wherein the therapeutic entity is a non-nucleoside inhibitor of HCV.

6. The method of claim 1, wherein the therapeutic entity is a compound of Formula (A).

7. The method of claim 1, wherein the therapeutic entity is Compound 1, Compound 2, or Compound 3.

8. A nucleotide probe comprising a sequence capable of detecting a mutation within HCV, wherein the mutation corresponds to 1) Q581E, A391V, M582L, or C432S within NS3 region, 2) V24A within NS4A region, 3) L4P, Q93, or L78T within NS4B region, 4) M416E441G, or V362A within NS5A region, or 5) C316Y, C445F, Y448H, or Y452H within NS5B region.

9. The nucleotide probe of claim 8, wherein the probe is labeled with a detectable group.

10. A cell line containing HCV replicon comprising at least one mutation selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, or 5) C316Y, C445F, Y448H, and Y452H within NS5B region.

11. An HCV replicon containing at least one mutation selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region.

12. A method for determining treatment for an HCV infection comprising detecting the presence or absence of a HCV mutation selected from the group consisting of 1) Q581 E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region.

13. The method of claim 12 comprising detecting the presence or absence of at least two HCV mutations selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region.

14. The method of claim 12 comprising detecting the presence or absence of at least three HCV mutations selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region.

15. An antibody capable of specifically binding to a HCV binding site containing at least one amino acid selected from the group consisting of 1) Q581, A391, M582, and C432 within NS3 region, 2) V24 within NS4A region, 3) L4, Q93, and L78 within NS4B region, 4) M416, E441, and V362 within NS5A region, and 5) C316, C445, Y448, and Y452 within NS5B region.

16. The antibody of claim 15, wherein the antibody is a monoclonal antibody.

17. The antibody of claim 15, wherein the antibody is a humanized antibody.

18. An antibody capable of specifically binding to an HCV epitope containing at least one mutation selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region.

19. The antibody of claim 18, wherein the antibody is a monoclonal antibody.

20. A method for screening potential therapeutic entities comprising contacting a testing entity with an HCV replicon comprising at least one mutation selected from the group consisting of 1) Q581E, A391V, M582L, and C432S within NS3 region, 2) V24A within NS4A region, 3) L4P, Q93, and L78T within NS4B region, 4) M416T, E441G, and V362A within NS5A region, and 5) C316Y, C445F, Y448H, and Y452H within NS5B region, and determining anti-HCV activity of the testing entity.

21. The method of claim 20, wherein the HCV replicon comprises at least one mutation selected from the group consisting of C316Y, C445F, Y448H, and Y452H within NS5B region.

22. The method of claim 20, wherein the HCV replicon comprises at least one mutation selected from the group consisting of V24A within NS4A region, M416T and E441G within NS5A region, and C316Y, C445F, Y448H, and Y452H within NS5B region.

23. A composition or method as described herein.
